(19) United States
(12) Patent Application Publication
(10) Pub. No.: US 2025/0099014 A1
(43) Pub. Date: Mar. 27, 2025

(54) METHOD AND SYSTEM FOR REAL-TIME CALIBRATION OF EAR-EEG DEVICE

(71) Applicant: Tata Consultancy Services Limited, Mumbai (IN)

(72) Inventors: Jayavardhana Rama GUBBI LAKSHMINARASIMHA, Bangalore (IN); Tanuja JAYAS, Bangalore (IN); Kartik MURALIDHARAN, Bangalore (IN); Adarsh ANAND, Bangalore (IN); Ramesh Kumar RAMAKRISHNAN, Bangalore (IN); Arpan PAL, Kolkata (IN)

(73) Assignee: Tata Consultancy Services Limited, Mumbai (IN)

(21) Appl. No.: 18/822,583

(22) Filed: Sep. 3, 2024

(30) Foreign Application Priority Data
Sep. 27, 2023 (IN) 202321065013

(51) Int. Cl. A61B 5/372; A61B 5/00; A61B 5/256

(52) U.S. Cl. CPC A61B 5/372; A61B 5/256; A61B 5/6815; A61B 5/6885; A61B 5/7221; A61B 5/7267; A61B 5/74; A61B 2560/0223; A61B 2560/045

(57) ABSTRACT

The embodiments of the present disclosure herein address unresolved problems of quality of signals in real time for wearables to provide optimal signals which can be used for brain signal based applications. Further, conventional techniques fail to provide real-time calibration of wearable devices, to understand the quality of the signals from the wearable device. Embodiments herein provide a method and system for a real-time calibration of one or more Electroencephalography (EEG) signals received from a wearable Ear-EEG device. The system is leveraging quality of signals in real time for wearables to provide optimal signals which can be used for early detection of neurodegenerative disease and brain-computer interface (BCI) applications. Further, the system is able to detect electrodes in the wearable device where the EEG signals have not been collected because the contact was not established.

### PRIORITY CLAIM

This U.S. patent application claims priority under 35 U.S.C. § 119 to Indian application number 202321065013, filed on Sep. 27, 2023. The entire content of the abovementioned application is incorporated herein by reference.

### TECHNICAL FIELD

The disclosure herein generally relates to the field of a signal processing, and more particularly, a method and system for a real-time calibration of one or more bioelectric signals received from a wearable Ear-EEG device.

### BACKGROUND

Electroencephalography (EEG) is a widely used noninvasive technique for measuring the electrical activity of the brain. The EEG has extensive applications in the study of brain functions in various domains like neurophysiology, brain-computer interface (BCI) and neuro-marketing. Lack of mobility, discomfort and strong decoding methods are a few obstacles in the monitoring of the brain activity. The main requirement for the interpretation and application of an EEG is good signal quality. Traditional approaches of assessing EEG signal quality involve visual inspection of the EEG time series for assessing any change in signal features. Further, the analysis of EEG signals for everyday in-situ applications is hindered by many challenges including artifacts induced from physiological and environmental sources as well as anatomical factors.

The acquisition of EEG signals from the scalp is very discomforting and does not have freedom of movement. Further, the EEG recording using ear wearables is a new modality which is more user friendly when compared to the existing scalp EEG. But the EEG signals recorded from these devices typically contain a lot of noise. Also, the EEG signals from some of the channels may not even get recorded because of lack of good skin-electrode connectivity.

Ear wearables are emerging technology and there is a lack of understanding of the EEG signals. Due to the nature of the brain signals, a standard template is not available against which the acquired EEG signals can be compared. So, it is important to collect Ear-EEG signal and calibrate it to get the proper signal, which would actually be Ear-EEG. Thus, it is important to check the quality of the EEG signal before using it for further downstream analysis. Visual inspection of signal is done to determine the presence of any kind of noise and to check if the signal was captured or not. For a consumer device, this approach is infeasible. Also, the inspection may differ from one expert to another. There is a lack of any particular method that can be used to check the quality of signals in real time for wearables to ensure optimal signal quality which can be used for applications such as early detection of neurodegenerative diseases and BCI. There is no such method present that can be used for the real-time calibration of such wearable devices, to understand the quality of the EEG signals from the device.

### SUMMARY

Embodiments of the present disclosure present technological improvements as solutions to one or more of the above-mentioned technical problems recognized by the inventors in conventional systems. For example, in one embodiment, a method for a real-time calibration of one or more signals received from a wearable Ear-EEG device is provided. The processor-implemented method includes receiving, via an input/output interface, a multitude of bioelectric signals of a user from a plurality of electrodes within a wearable Ear-EEG device, wherein the plurality of electrodes includes a ground electrode and a reference electrode.

Further, the processor-implemented method comprises collecting a set of context specific features from a mobile application within a mobile device and determining a contact of each of the plurality of electrodes with the user in order to receive the multitude of bioelectric signals based on a set of impedance values, wherein the set of impedance values is an impedance between each of the plurality of electrodes and a skin-body interface of the user. Furthermore, the processor-implemented method comprises verifying the received multitude of bioelectric signals from the plurality of electrodes based on a flat channel detection technique to have a predefined optimum voltage level, wherein one or more electrodes of the plurality of electrodes are removed corresponding to the multitude of bioelectric signals having a predefined saturated voltage level using the flat channel detection technique. Further, the processor-implemented method comprises selecting a set of electrodes of the plurality of electrodes which receives a set of EEG signals based on one or more characteristics of the multitude of bioelectric signals and the collected set of context specific features, wherein the one or more characteristics of the multitude of bioelectric signals is at least one of (1) in time domain, or (2) in frequency domain, or (3) in both time and frequency domain.

Furthermore, the processor-implemented method comprises checking the selected set of electrodes of the plurality of electrodes to send an alert to the user to recalibrate the Ear-EEG device if the selected set of electrodes are lesser than a predefined minimum number, evaluating a quality index value of each of the received set of EEG signals based on a predefined threshold value for the mobile application using a pre-trained machine learning model, wherein one or more characteristics of the received multitude of bioelectric signals along with the set of application specific features are used to determine the signal quality of the received set of EEG signals, and classifying the selected set of electrodes based on the evaluated quality index value of the set of EEG signals and a set of artifacts of the set of EEG signals, wherein a continuous detection of the set of artifacts of the received EEG signals is carried out. Finally, calibrating again the ear-EEG device based on the evaluated quality index value of the set of EEG signals received from the selected one or more electrodes.

In another aspect, a system for real-time calibration of one or more EEG signals received from a wearable Ear-EEG device is provided. The system comprises a memory storing a plurality of instructions and one or more Input/Output (I/O) interfaces to receive a multitude of bioelectric signals of a user from a plurality of electrodes within a wearable Ear-EEG device, wherein the plurality of electrodes includes a ground electrode and a reference electrode. Further, the system comprises one or more hardware processors coupled to the memory via the one or more I/O interfaces, wherein the one or more hardware processors are configured by the instructions to execute a plurality of modules of the system.

Further, the system is configured to collect a set of context specific features from a mobile application within a mobile device and determine a contact of each of the plurality of electrodes with the user in order to receive the multitude of bioelectric signals based on a set of impedance values. Wherein the set of context specific features are based on an initial context from day-to-day activities of the user collected automatically from the wearable Ear-EEG device and the wherein the set of impedance values is an impedance between each of the plurality of electrodes and a skin-body interface of the user. The received multitude of bioelectric signals are verified from the plurality of electrodes based on a flat channel detection technique to have a predefined optimum voltage level, wherein one or more electrodes of the plurality of electrodes are removed corresponding to the multitude of bioelectric signals having a predefined saturated voltage level using the flat channel detection technique. Further, a set of electrodes of the plurality of electrodes which receives a set of EEG signals are selected based on one or more characteristics of the multitude of bioelectric signals and the collected set of context specific features, wherein the one or more characteristics of the multitude of bioelectric signals is at least one of (1) in time domain, or (2) in frequency domain, or (3) in both time and frequency domain.

Furthermore, the system is configured to check the selected set of electrodes of the plurality of electrodes to send an alert to the user to recalibrate the Ear-EEG device if the selected set of electrodes are lesser than a predefined minimum number. Further, the system is configured to evaluate a quality index value of each of the received set of EEG signals based on a predefined threshold value for the mobile application using a pre-trained machine learning model and the selected set of electrodes are classified based on the evaluated quality index value of the set of EEG signals and a set of artifacts of the set of EEG signals. A continuous detection of the set of artifacts of the received EEG signals is carried out, wherein one or more characteristics of the received multitude of bioelectric signals along with the set of application specific features are used to determine the signal quality of the received set of EEG signals. Finally, the ear-EEG device is calibrated again based on the evaluated quality index value of the set of EEG signals received from the selected one or more electrodes.

In yet another aspect, there are provided one or more non-transitory machine-readable information storage mediums comprising one or more instructions, which when executed by one or more hardware processors causes a method for a real-time calibration of one or more EEG signals received from a wearable Ear-EEG device is provided. Further, the processor-implemented method comprises collecting a set of context specific features from a mobile application within a mobile device and determining a contact of each of the plurality of electrodes with the user in order to receive the multitude of bioelectric signals based on a set of impedance values, wherein the set of impedance values is an impedance between each of the plurality of electrodes and a skin-body interface of the user. Furthermore, the processor-implemented method comprises verifying the received multitude of bioelectric signals from the plurality of electrodes based on a flat channel detection technique to have a predefined optimum voltage level, wherein one or more electrodes of the plurality of electrodes are removed corresponding to the multitude of bioelectric signals having a predefined saturated voltage level using the flat channel detection technique. Further, the processor-implemented method comprises selecting a set of electrodes of the plurality of electrodes which receives a set of EEG signals based on one or more characteristics of the multitude of bioelectric signals and the collected set of context specific features, wherein the one or more characteristics of the multitude of bioelectric signals is at least one of (1) in time domain, or (2) in frequency domain, or (3) in both time and frequency domain.

Furthermore, the processor-implemented method comprises checking the selected set of electrodes of the plurality of electrodes to send an alert to the user to recalibrate the Ear-EEG device if the selected set of electrodes are lesser than a predefined minimum number, evaluating a quality index value of each of the received set of EEG signals based on a predefined threshold value for the mobile application using a pre-trained machine learning model, wherein one or more characteristics of the received multitude of bioelectric signals along with the set of application specific features are used to determine the signal quality of the received set of EEG signals, and classifying the selected set of electrodes based on the evaluated quality index value of the set of EEG signals and a set of artifacts of the set of EEG signals, wherein a continuous detection of the set of artifacts of the received EEG signals is carried out. Finally, calibrating again the Ear-EEG device based on the evaluated quality index value of the set of EEG signals received from the selected one or more electrodes.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles:

FIG. 1 illustrates a system for real-time calibration of one or more EEG signals received from a wearable Ear-EEG device, according to some embodiments of the present disclosure.

FIG. 2 is a functional block diagram to illustrate a system for real-time calibration of one or more EEG signals received from a wearable Ear-EEG device, according to some embodiments of the present disclosure.

FIGS. 3A and 3B (collectively referred as FIG. 3) are an exemplary flow diagram illustrating a processor-implemented method for real-time calibration of one or more EEG signals received from a wearable Ear-EEG device, according to some embodiments of the present disclosure.

### DETAILED DESCRIPTION

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the scope of the disclosed embodiments.

Electroencephalography (EEG) is a widely used noninvasive technique for measuring the electrical activity of the brain. The EEG has extensive applications in the study of brain functions in various domains like neurophysiology, brain-computer interface (BCI) and neuro-marketing. Lack of mobility, discomfort and strong decoding methods are a few obstacles in the monitoring of the brain activity. An Ear-EEG is more user friendly and portable in comparison to the existing scalp EEG. This form of wearable EEG finds various use cases in everyday life for detection/monitoring sleep, stress, emotion, and cognition.

The main requirement for the interpretation and application of an EEG is good signal quality. However, there is a lack of research on this topic in relation to newer mobile EEG devices, in particular ear worn devices. Traditional approaches of assessing EEG signal quality involve visual inspection of the EEG time series for assessing any change in signal features. Analysis of EEG signals for everyday in-situ applications is hindered by many challenges including artifacts induced from physiological and environmental sources as well as anatomical factors.

Recent studies have proposed analytical frameworks for the assessment of scalp EEG quality and identification of artifacts using rule-based methods. These methods are typically used in an offline processing manner, employed before signal analysis when abundant signals are available. With the advent of wearable devices, it is important to build techniques that work on short term signals giving us the ability of intervention based on the signal quality. An Ear-EEG is a new class of wearable device that requires assessment and calibration with short term signals periodically. To support this new modality, a detailed study of scalp and Ear-EEG signal is conducted from the perspective of different measures as well as time duration. An algorithm is developed to identify the epochs with electrooculography (EOG) and electromyography (EMG) artifacts in the Ear-EEG using a set of metrics based on the characteristics of EEG. Thresholds of these metrics are determined by training on a dataset containing synchronous ear and scalp EEG with good results. Embodiments herein provide a method and system for a real-time calibration of one or more EEG signals received from a wearable Ear-EEG device. The system is leveraging quality of signals in real time for wearables to provide optimal signals which can be used for BCI applications. Further, the system is able to detect sensors (electrodes) in the wearable device where the signal has not been collected because the contact was not established.

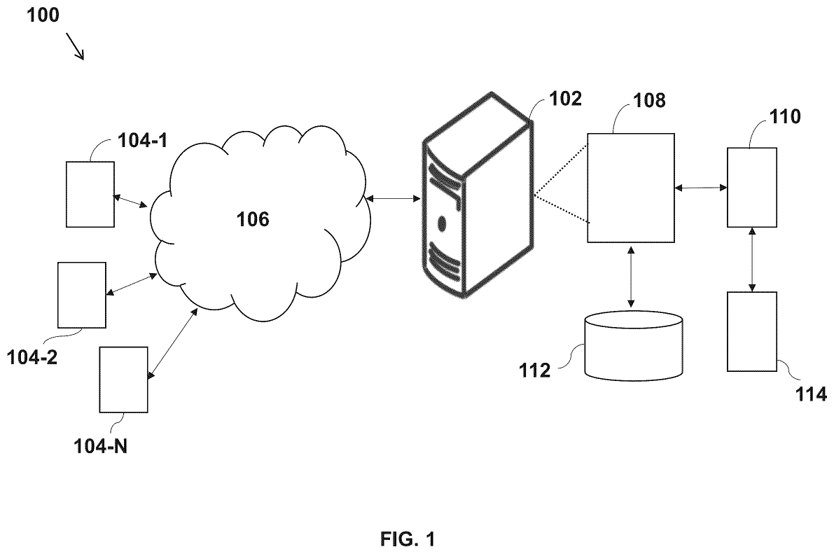

Referring now to the drawings, and more particularly to FIG. 1 through FIG. 3B, where similar reference characters denote corresponding features consistently throughout the figures, there are shown preferred embodiments, and these embodiments are described in the context of the following exemplary system and/or method.

FIG. 1 illustrates a block diagram of system for a real-time calibration of one or more EEG signals received from a wearable Ear-EEG device, in accordance with an example embodiment. Although the present disclosure is explained considering that the system 100 is implemented on a server, it may be understood that the system 100 may comprise one or more computing devices 102, such as a laptop computer, a desktop computer, a notebook, a workstation, a cloud-based computing environment and the like. It will be understood that the system 100 may be accessed through one or more input/output interfaces 104-1, 104-2 . . . 104-N, collectively referred to as I/O interface 104. Examples of the I/O interface 104 may include, but are not limited to, a user interface, a portable computer, a personal digital assistant, a handheld device, a smartphone, a tablet computer, a workstation, and the like. The I/O interface 104 is communicatively coupled to the system 100 through a network 106.

In an embodiment, the network 106 may be a wireless or a wired network, or a combination thereof. In an example, the network 106 can be implemented as a computer network, as one of the different types of networks, such as virtual private network (VPN), intranet, local area network (LAN), wide area network (WAN), the internet, and such. The network 106 may either be a dedicated network or a shared network, which represents an association of the different types of networks that use a variety of protocols, for example, Hypertext Transfer Protocol (HTTP), Transmission Control Protocol/Internet Protocol (TCP/IP), and Wireless Application Protocol (WAP), to communicate with each other. Further, the network 106 may include a variety of network devices, including routers, bridges, servers, computing devices, storage devices. The network devices within the network 106 may interact with the system 100 through communication links.

The system 100 supports various connectivity options such as BLUETOOTH®, USB, ZigBee, and other cellular services. The network environment enables connection of various components of the system 100 using any communication link including Internet, WAN, MAN, and so on. In an exemplary embodiment, the system 100 is implemented to operate as a stand-alone device. In another embodiment, the system 100 may be implemented to work as a loosely coupled device to a smart computing environment. Further, the system 100 comprises at least one memory 110 with a plurality of instructions, one or more databases 112, and one or more hardware processors 108 which are communicatively coupled with the at least one memory to execute a plurality of modules 114 therein. The components and functionalities of the system 100 are described further in detail.

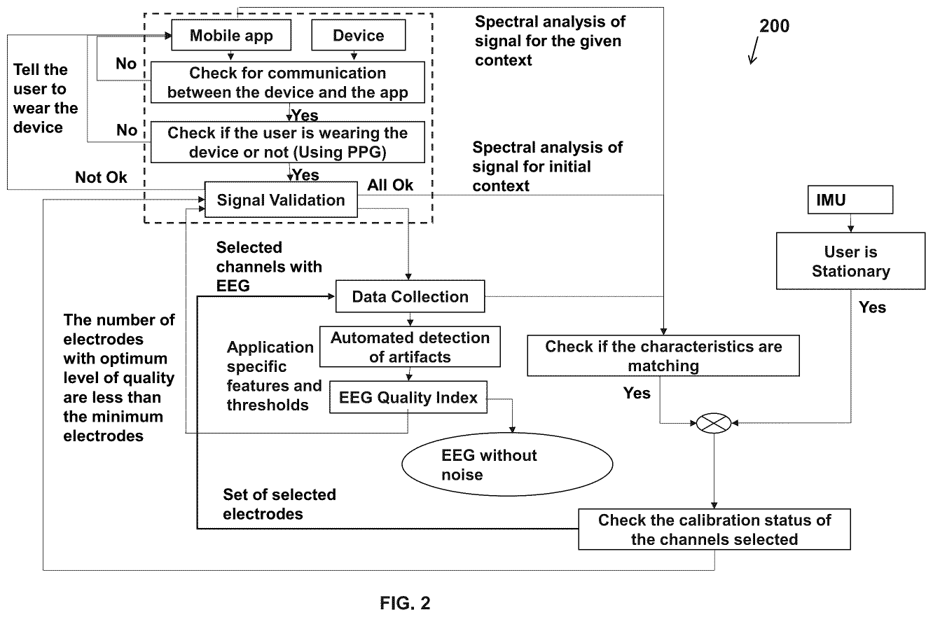

FIG. 2 is a functional block diagram 200 to illustrate the system 100 for real-time calibration of one or more signals received from a wearable Ear-EEG device, according to some embodiments of the present disclosure. In one embodiment, the system 100 is configured to measure a pair of impedance values between a plurality of electrodes and the human body to identify a set of electrodes which receives as well as do not receive the multitude of signals including EEG. Based on the pair of impedance values and a context from the day-to-day activities by a user, the set of electrodes where the EEG signal is being received is selected for signal extraction without disturbing the user. Further, the system 100 is configured to verify whether the voltage levels of the multitude of bioelectric signals received from the plurality of electrodes is attaining a saturation and based on the voltage level, those electrodes attaining saturation levels are removed from the plurality of electrodes.

In yet another embodiment, the system 100 is configured for selection of electrodes where the presence of EEG is determined using a set of context specific features extracted from a mobile application. The set of context specific features may vary according to the stimulus from activities of the user. Further, the system 100 is configured to estimate a quality index using a set of metrics based on characteristics of the EEG signal.

In another aspect, a framework is created to compute the quality metrics of an epoch and check if the metrics are in range of the thresholds of the EEG signal. The features evaluated in this section are statistical parameters which represent the complexity and strength of the Ear-EEG signal in both time and frequency domain. Further, the disclosure includes another embodiment for an artifact detection to identify and remove the artifacts in the Ear-EEG signals to get EEG signal in the end.

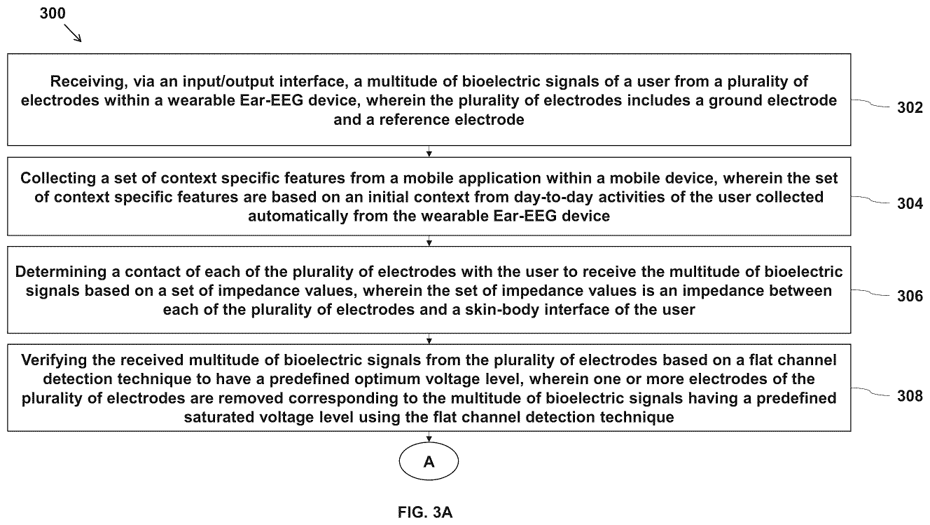

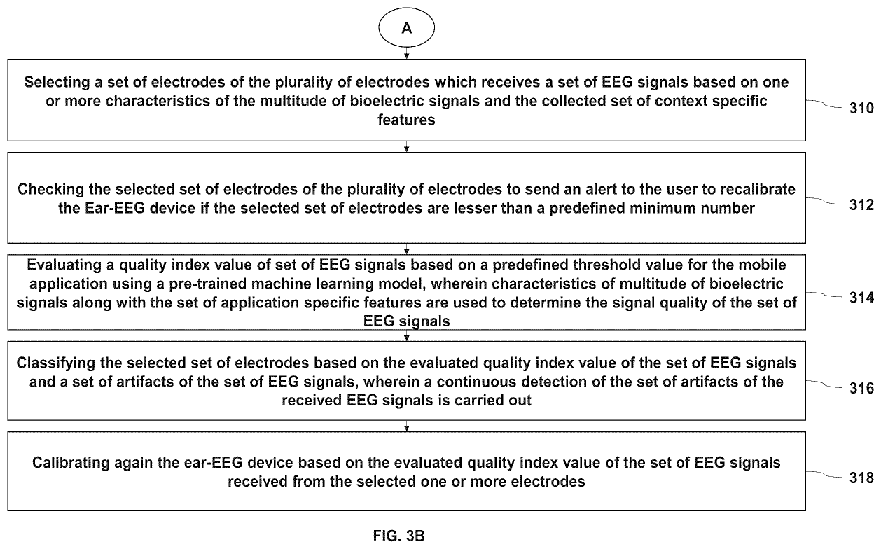

FIGS. 3A and 3B (collectively referred as FIG. 3) are a flow diagram illustrating a processor-implemented method 300 for a real-time calibration of one or more EEG signals received from a wearable Ear-EEG device implemented by the system 100 of FIG. 1. Functions of the components of the system 100 are now explained through steps of flow diagram in FIG. 3A and 3B, according to some embodiments of the present disclosure.

Initially, at step 302 of the processor-implemented method 300, the one or more hardware processors 108 are configured by the programmed instructions to receive, via an input/output interface, a multitude of bioelectric signals of a user from a plurality of electrodes within a wearable Ear-EEG device, wherein the plurality of electrodes includes a ground electrode and a reference electrode.

At the next step 304 of the processor-implemented method 300, the one or more hardware processors 108 are configured by the programmed instructions to a set of context specific features from a mobile application within a mobile device. The set of context specific features are based on an initial context from day-to-day activities of the user collected automatically from the wearable Ear-EEG device. An alert to wear the Ear-EEG device is sent to the user in case of an absence of the prompt message from the mobile application.

At the next step 306 of the processor-implemented method 300, the one or more hardware processors 108 are configured by the programmed instructions to determine a contact of each of the plurality of electrodes with the user in order to receive the multitude of bioelectric signals based on a set of impedance values. The set of impedance values is an impedance between each of the electrodes and skin-body interface of the user. The set of impedance values between the user (skin-body) and each of the plurality of electrodes should be a minimum (low impedance conductive path) compared to a predefined threshold value of impedance, to receive the multitude of bioelectric signals. A set of electrodes with low impedance values are selected to receive the multitude of bioelectric signals based on the set of impedance values measured.

At the next step 308 of the processor-implemented method 300, the one or more hardware processors 108 are configured by the programmed instructions to verify the received multitude of bioelectric signals from the plurality of electrodes based on a flat channel detection technique to have a predefined optimum voltage level. The one or more electrodes of the plurality of electrodes are removed corresponding to the multitude of bioelectric signals having a predefined saturated voltage level using the flat channel detection technique. The received multitude of bioelectric signals within the optimum voltage levels are collected for processing e.g., the amplitude of the signal should be between +/−50 micro volts. The received multitude of bioelectric signals with the saturated voltage level +/−500 micro volts are not actual EEG and hence rejected using the flat channel detection process.

At the next step 310 of the processor-implemented method 300, the one or more hardware processors 108 are configured by the programmed instructions to select a set of electrodes of the plurality of electrodes which receives a set of EEG signals based on one or more characteristics of the multitude of bioelectric signals and the collected set of context specific features. The one or more characteristics of the multitude of bioelectric signals is at least one of (1) in time domain, or (2) in frequency domain, or (3) in both time and frequency domain. The channel selection technique determines whether the received multitude of bioelectric signals are EEG or not. The selected set of EEG signals are separated from the received multitude of bioelectric signals using a predefined threshold voltage level based on a spatial characteristic (location of the electrode in the ear including concha or ear canal) as well as context specific features (the activity being performed by the user such as watching video, listening to music, playing a video game, etc.).

At the next step 312 of the processor-implemented method 300, the one or more hardware processors 108 are configured by the programmed instructions to check the selected set of electrodes of the plurality of electrodes to send an alert to the user to recalibrate the Ear-EEG device if the selected set of electrodes are lesser than a predefined minimum number. For a real-time check of the set of electrodes collecting the set of continuous EEG signal with quality levels for the specified application, a continuous check on the spectral characteristics of the set of EEG signals for the given context (Listening to music, watching a video, playing game) is done. Wherein, if the set of electrodes which receives the set of EEG signals are minimum, a user alert is sent to recalibrate the Ear-EEG device.

The status of the electrodes where the signal is being collected is checked when the spectral characteristics of the EEG signal for the given context is similar to the spectral characteristics of the initial context and at the same time the user is stationary. The information about the context and physical activity of the user is being collected from a sensor array (e.g., Inertial Measurement Unit (IMU)), wherein if the minimum number of electrodes checked when the user is stationary is achieved, the signal collection is continued if the number of electrodes collecting EEG is more than the required minimum number.

At the next step 314 of the processor-implemented method 300, the one or more hardware processors 108 are configured by the programmed instructions to evaluate a quality index value of each of the received set of EEG signals based on a predefined threshold value for the mobile application using a pre-trained machine learning model. Wherein, one or more characteristics of the received multitude of bioelectric signals along with the set of application specific features are used to determine the signal quality of the received set of EEG signals. Herein, a set of quality metrics based on characteristics of the received EEG signal is defined to measure the quality of the signal. The quality metric consists of features like: Signal-to-noise ratio (SNR), Root mean square (RMS) of the amplitude, Kurtosis of the amplitude, Skewness of the amplitude, mean of the power, zero crossing rate (ZCR), Spectral entropy, Maximum gradient, Auto-correlation function (ACF). The values of these metric will reflect the characteristics of the signal in both frequency and time domain.

In another aspect, a framework is created to compute the set of quality metrics for each of the set of EEG signal at an instant and check if the set of metrics are in range of a threshold. The thresholds for the quality index are application specific. The computed set of quality metrics is based on characteristics of the selected EEG signal defined to measure the quality of the signal. The set of quality metrics are constructed based on features which represent the complexity and strength of the set of EEG signals in both time and frequency domain. A spatial characteristic of the received multitude of bioelectric signals along with the set of context specific features are used to determine a signal quality of the received multitude of bioelectric signals. The signal quality varies according to the set of context specific features.

At the next step 316 of the processor-implemented method 300, the one or more hardware processors 108 are configured by the programmed instructions to classify the selected set of electrodes based on the evaluated quality index value of the set of EEG signals and a set of artifacts of the set of EEG signals, whether the set of artifacts (such as EOG and EMG) are present in them or not. Wherein a continuous detection of artifacts in the EEG signal collected is carried out and along with the quality of EEG signal is checked using an EEG Quality Index (EQI). The set of EEG signals are labelled as with the set of artifacts and without artifacts using machine learning algorithms. Tree based machine learning algorithms such as Decision Tree, Random Forest, Gradient Boost, XG Boost, Light Gradient boosting, are trained to classify the EEG signals. The defined metrics are calculated for a given segment of the EEG signals in four different bands of the signal namely, alpha, beta, gamma, and delta and for the raw signal. The evaluation of metrics in different bands captures the features of the signal in different spatial regions. This set of metrics for each segment is normalized and later is fed to a machine learning classifier which labels that particular segment with or without artifact.

Finally, at the last step 318 of the processor-implemented method 300, the one or more hardware processors 108 are configured by the programmed instructions to re-calibrate the ear-EEG device based on the evaluated quality index value of the set of EEG signals received from the selected one or more electrodes.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments of present disclosure herein address unresolved problems of quality of signals in real time for wearables to provide optimal signals which can be used for BCI applications. Further, there is no such method present that can be used for the real-time calibration of wearable devices to understand the quality of the EEG signals from the wearable device. Embodiments herein provide a method and system for a real-time calibration of one or more EEG signals received from a wearable Ear-EEG device. The system is leveraging quality of signal in real time for wearables to provide optimal signals which can be used for BCI applications. Further, the system is able to detect sensors (electrodes) in the wearable where the signal has not been collected because the contact was not established.

It is to be understood that the scope of the protection is extended to such a program and in addition to a computer-readable means having a message therein; such computer-readable storage means contain program-code means for implementation of one or more steps of the method, when the program runs on a server or mobile device or any suitable programmable device. The hardware device can be any kind of device which can be programmed including e.g., any kind of computer like a server or a personal computer, or the like, or any combination thereof. The device may also include means which could be e.g., hardware means like e.g., an application-specific integrated circuit (ASIC), a field-programmable gate array (FPGA), or a combination of hardware and software means, e.g., an ASIC and an FPGA, or at least one microprocessor and at least one memory with software processing components located therein. Thus, the means can include both hardware means, and software means. The method embodiments described herein could be implemented in hardware and software. The device may also include software means. Alternatively, the embodiments may be implemented on different hardware devices, e.g., using a plurality of CPUs.

The embodiments herein can comprise hardware and software elements. The embodiments that are implemented in software include but are not limited to, firmware, resident software, microcode, etc. The functions performed by various components described herein may be implemented in other components or combinations of other components. For the purposes of this description, a computer-usable or computer readable medium can be any apparatus that can comprise, store, communicate, propagate, or transport the program for use by or in connection with the instruction execution system, apparatus, or device.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope of disclosed embodiments being indicated by the following claims.

What is claimed is:

1. A processor-implemented method comprising:
receiving, via an Input/Output (I/O) interface, a multitude of bioelectric signals of a user from a plurality of electrodes within a wearable Ear-Electroencephalography (EEG) device, wherein the plurality of electrodes includes a ground electrode and a reference electrode;
collecting, via one or more hardware processors, a set of context specific features from a mobile application within a mobile device, wherein the set of context specific features are based on an initial context from day-to-day activities of the user collected automatically from the wearable Ear-EEG device;
determining, via the one or more hardware processors, a contact of each of the plurality of electrodes with the user in order to receive the multitude of bioelectric signals based on a set of impedance values, wherein the set of impedance values is an impedance between each of the plurality of electrodes and a skin-body interface of the user;
verifying, via the one or more hardware processors, the received multitude of bioelectric signals from the plurality of electrodes based on a flat channel detection technique to have a predefined optimum voltage level, wherein one or more electrodes of the plurality of electrodes are removed pertaining to the multitude of bioelectric signals having a predefined saturated voltage level using the flat channel detection technique;
selecting, via the one or more hardware processors, a set of electrodes of the plurality of electrodes which receives a set of EEG signals based on one or more characteristics of the multitude of bioelectric signals and the collected set of context specific features, wherein the one or more characteristics of the multitude of bioelectric signals is at least one of (1) in time domain, or (2) in frequency domain, or (3) in both time and frequency domain;
checking, via the one or more hardware processors, the selected set of electrodes of the plurality of electrodes to send an alert to the user to recalibrate the Ear-EEG device if the selected set of electrodes is lesser than a predefined minimum number;
evaluating, via the one or more hardware processors, a quality index value of each of the received set of EEG signals based on a predefined threshold value for the mobile application using a pre-trained machine learning model, wherein one or more characteristics of the received multitude of bioelectric signals along with the set of application specific features are used to determine the signal quality of the received set of EEG signals;
classifying, via the one or more hardware processors, the selected set of electrodes based on the evaluated quality index value of the set of EEG signals and a set of artifacts of the set of EEG signals, wherein a continuous detection of the set of artifacts of the received EEG signals is carried out; and
re-calibrating, via the one or more hardware processors, the ear-EEG device based on the evaluated quality index value of the set of EEG signals received from the selected one or more electrodes.

2. The processor-implemented method of claim 1, wherein the initial context is a prompt message from the mobile application to the Ear-EEG device via the mobile device, and wherein an alert to wear the Ear-EEG device is sent to the user in case of an absence of the prompt message from the mobile application.

3. The processor-implemented method of claim 1, wherein the set of impedance values between the user skin-body and each of the plurality of electrodes is a predefined minimum low impedance conductive path compared to a predefined threshold value of impedance, in order to receive the multitude of bioelectric signals.

4. The processor-implemented method of claim 1, wherein the quality index value of each of the received set of EEG signals varies according to the context specific features of the mobile application.

5. The processor-implemented method of claim 1, wherein a framework is created to compute a set of quality metrics for each of the selected one or more electrodes receiving the set of EEG signals at an instant and check if the set of quality metrics are in range of the predefined threshold value, and wherein the predefined threshold values for the quality index are an application specific.

6. The processor-implemented method of claim 1, wherein:
if the quality index value drops below a predefined lowest threshold for the given mobile application by the user for a minimum number of electrodes, an EEG signal validation and an electrode selection are done again; and
if the quality index value is less than the predefined threshold value the electrode selection is carried out again.

7. A system comprising:
a memory storing instructions;
one or more Input/Output (I/O) interfaces; and
one or more hardware processors coupled to the memory via the one or more I/O interfaces, wherein the one or more hardware processors are configured by the instructions to:
receive a multitude of bioelectric signals of a user from a plurality of electrodes within a wearable Ear-Electroencephalography (EEG) device, wherein the plurality of electrodes includes a ground electrode and a reference electrode;
collect a set of context specific features from a mobile application within a mobile device, wherein the set of context specific features are based on an initial context from day-to-day activities of the user collected automatically from the wearable Ear-EEG device;
determine a contact of each of the plurality of electrodes with the user in order to receive the multitude of bioelectric signals based on a set of impedance values, wherein the set of impedance values is an impedance between each of the plurality of electrodes and a skin-body interface of the user;
verify the received multitude of bioelectric signals from the plurality of electrodes based on a flat channel detection technique to have a predefined optimum voltage level, wherein one or more electrodes of the plurality of electrodes are removed corresponding to the multitude of bioelectric signals having a predefined saturated voltage level using the flat channel detection technique;
select a set of electrodes of the plurality of electrodes which receives a set of EEG signals based on one or more characteristics of the multitude of bioelectric signals and the collected set of context specific features, wherein the one or more characteristics of the multitude of bioelectric signals is at least one of (1) in time domain, or (2) in frequency domain, or (3) in both time and frequency domain;
check the selected set of electrodes of the plurality of electrodes to send an alert to the user to recalibrate the Ear-EEG device if the selected set of electrodes are lesser than a predefined minimum number;
evaluate a quality index value of each of the received set of EEG signals based on a predefined threshold value for the mobile application using a pre-trained machine learning model, wherein one or more characteristics of the received multitude of bioelectric signals along with the set of application specific features are used to determine the signal quality of the received set of EEG signals;
classify the selected set of electrodes based on the evaluated quality index value of the set of EEG signals and a set of artifacts of the set of EEG signals, wherein a continuous detection of the set of artifacts of the received EEG signals is carried out; and
re-calibrate the ear-EEG device based on the evaluated quality index value of the set of EEG signals received from the selected one or more electrodes.

8. The system of claim 7, wherein the initial context is a prompt message from the mobile application to the Ear-EEG device via the mobile device, wherein an alert to wear the Ear-EEG device is sent to the user in case of an absence of the prompt message from the mobile application.

9. The system of claim 7, wherein the set of impedance values between the user skin-body and each of the plurality of electrodes is a predefined minimum low impedance conductive path compared to a predefined threshold value of impedance, in order to receive the multitude of bioelectric signals.

10. The system of claim 7, wherein the quality index value of each of the received set of EEG signals varies according to the context specific features of the mobile application.

11. The system of claim 7, wherein a framework is created to compute a set of quality metrics for each of the selected one or more electrodes receiving the set of EEG signals at an instant and check if the set of quality metrics are in range of the predefined threshold value, wherein the predefined threshold values for the quality index are an application specific.

12. The system of claim 7, wherein:
if the quality index value drops below a predefined lowest threshold for the given mobile application by the user for a minimum number of electrodes, an EEG signal validation and an electrode selection are done again; and
if the quality index value is less than the predefined threshold value the electrode selection is carried out again.

13. One or more non-transitory machine-readable information storage mediums comprising one or more instructions which when executed by one or more hardware processors cause:
receiving, via an Input/Output (I/O) interface, a multitude of bioelectric signals of a user from a plurality of electrodes within a wearable Ear-Electroencephalography (EEG) device, wherein the plurality of electrodes includes a ground electrode and a reference electrode;
collecting a set of context specific features from a mobile application within a mobile device, wherein the set of context specific features are based on an initial context from day-to-day activities of the user collected automatically from the wearable Ear-EEG device and the initial context is a prompt message from the mobile application to the Ear-EEG device via the mobile device, and wherein an alert to wear the Ear-EEG device is sent to the user in case of an absence of the prompt message from the mobile application;
determining a contact of each of the plurality of electrodes with the user in order to receive the multitude of bioelectric signals based on a set of impedance values, wherein the set of impedance values is an impedance between each of the plurality of electrodes and a skin-body interface of the user and the set of impedance values between the user skin-body and each of the plurality of electrodes is a predefined minimum low impedance conductive path compared to a predefined threshold value of impedance, in order to receive the multitude of bioelectric signals;
verifying the received multitude of bioelectric signals from the plurality of electrodes based on a flat channel detection technique to have a predefined optimum voltage level, wherein one or more electrodes of the plurality of electrodes are removed pertaining to the multitude of bioelectric signals having a predefined saturated voltage level using the flat channel detection technique;
selecting a set of electrodes of the plurality of electrodes which receives a set of EEG signals based on one or more characteristics of the multitude of bioelectric signals and the collected set of context specific features, wherein the one or more characteristics of the multitude of bioelectric signals is at least one of (1) in time domain, or (2) in frequency domain, or (3) in both time and frequency domain;
checking the selected set of electrodes of the plurality of electrodes to send an alert to the user to recalibrate the Ear-EEG device if the selected set of electrodes is lesser than a predefined minimum number;
evaluating a quality index value of each of the received set of EEG signals based on a predefined threshold value for the mobile application using a pre-trained machine learning model, wherein one or more characteristics of the received multitude of bioelectric signals along with the set of application specific features are used to determine the signal quality of the received set of EEG signals, and the quality index value of each of the received set of EEG signals varies according to the context specific features of the mobile application;
classifying the selected set of electrodes based on the evaluated quality index value of the set of EEG signals and a set of artifacts of the set of EEG signals, wherein a continuous detection of the set of artifacts of the received EEG signals is carried out; and
re-calibrating the ear-EEG device based on the evaluated quality index value of the set of EEG signals received from the selected one or more electrodes.

14. The one or more non-transitory machine-readable information storage mediums of claim 13, wherein the initial context is a prompt message from the mobile application to the Ear-EEG device via the mobile device, and wherein an alert to wear the Ear-EEG device is sent to the user in case of an absence of the prompt message from the mobile application.

15. The one or more non-transitory machine-readable information storage mediums of claim 13, wherein the set of impedance values between the user skin-body and each of the plurality of electrodes is a predefined minimum low impedance conductive path compared to a predefined threshold value of impedance, in order to receive the multitude of bioelectric signals.

16. The one or more non-transitory machine-readable information storage mediums of claim 13, wherein the quality index value of each of the received set of EEG signals varies according to the context specific features of the mobile application.

17. The one or more non-transitory machine-readable information storage mediums of claim 13, wherein a framework is created to compute a set of quality metrics for each of the selected one or more electrodes receiving the set of EEG signals at an instant and check if the set of quality metrics are in range of the predefined threshold value, and wherein the predefined threshold values for the quality index are an application specific.

18. The one or more non-transitory machine-readable information storage mediums of claim 13, wherein:
if the quality index value drops below a predefined lowest threshold for the given mobile application by the user for a minimum number of electrodes, an EEG signal validation and an electrode selection are done again; and
if the quality index value is less than the predefined threshold value the electrode selection is carried out again.
